(19) United States
(12) Patent Application Publication
(10) Pub. No.: US 2024/0303807 A1
(43) Pub. Date: Sep. 12, 2024

(54) INSPECTION ASSISTANCE DEVICE, INSPECTION ASSISTANCE METHOD, AND RECORDING MEDIUM

(71) Applicant: NEC Corporation, Minato-ku, Tokyo (JP)

(72) Inventors: Ayaka AMAKAWA, Tokyo (JP); Tomoharu Klyuna, Tokyo (JP); Maki Sano, Tokyo (JP)

(73) Assignee: NEC Corporation, Minato-ku, Tokyo (JP)

(21) Appl. No.: 18/268,349

(22) PCT Filed: Mar. 25, 2021
(86) PCT No.: PCT/JP2021/012495

§ 371 Date: Jun. 20, 2023

(51) Int. Cl. G06T 7/00; G06T 7/11

(52) U.S. Cl. CPC G06T 7/0012; G06T 7/11; G06T 2207/20081; G06T 2207/30096

(57) ABSTRACT

Provided is a technology for reducing calculation cost for identifying a region that has a high tumor content ratio and is used for gene panel testing. This testing support device comprises an acquisition means that acquires a pathological specimen image, a region reception means that receives designation of a region for the acquired pathological specimen image, a detection means that detects the position of a tumor cell and the position of a non-tumor cell in the designated region of the pathological specimen image, and a calculation means that calculates a tumor content ratio for each divided region, which is obtained through division a predetermined size, on the basis of the detected position of a tumor cell and the detected position of a non-tumor cell.

### TECHNICAL FIELD

The present invention relates to an inspection assistance device, an inspection assistance system, an inspection assistance method, and a recording medium.

### BACKGROUND ART

In recent years, a genetic panel test has been widely used as a cancer examination method. In the genetic panel test, it is necessary to cut off a region having a high tumor cell content rate from a specimen and use the region for the test.

In the technique described in PTL 1, description is made in which a state of a cell or a tissue in a pathological image is identified using a training model machine trained with a large number of samples of the pathological image, and the identification result is visualized.

### CITATION LIST

### Patent Literature

PTL 1: JP 2018-044806 A

### SUMMARY OF INVENTION

### Technical Problem

In order to use the pathology specimen for the genetic panel test, it is necessary to identify a region having a high tumor cell content rate from the pathological image. However, when processing is performed on the entire image as in PTL 1, there is a problem that the calculation cost of the identification processing is high. The present invention has been made in view of the above problems, and an object of the present invention is to provide a technique for reducing a calculation cost for identifying a region having a high tumor cell content rate for a genetic panel test.

### Solution to Problem

According to a first point of view of the present invention, an inspection assistance device including an acquisition means configured to acquire a pathology specimen image, a region reception means configured to receive designation of a region for the acquired pathology specimen image, a detection means configured to detect a position of a tumor cell and a position of a non-tumor cell in the pathology specimen image in the designated region, and a calculation means configured to calculate a tumor cell content rate for each divided region obtained by division by a predetermined size based on the detected position of the tumor cell and the detected position of the non-tumor cell is provided.

According to a second point of view of the present invention, an inspection assistance system including an acquisition means configured to acquire a pathology specimen image, a region reception means configured to receive designation of a region for the acquired pathology specimen image, a detection means configured to detect a position of a tumor cell and a position of a non-tumor cell in the pathology specimen image in the designated region, and a calculation means configured to calculate a tumor cell content rate for each divided region obtained by division by a predetermined size based on the detected position of the tumor cell and the detected position of the non-tumor cell is provided.

According to a third point of view of the present invention, an inspection assistance method including acquiring a pathology specimen image, receiving designation of a region for the acquired pathology specimen image, detecting a position of a tumor cell and a position of a non-tumor cell in the pathology specimen image in the designated region, and calculating a tumor cell content rate for each divided region obtained by division by a predetermined size based on the detected position of the tumor cell and the detected position of the non-tumor cell is provided.

According to a fourth point of view of the present invention, a recording medium storing a program for causing a computer to function as an acquisition means configured to acquire a pathology specimen image, a region reception means configured to receive designation of a region for the acquired pathology specimen image, a detection means configured to detect a position of a tumor cell and a position of a non-tumor cell in the pathology specimen image in the designated region, and a calculation means configured to calculate a tumor cell content rate for each divided region obtained by division by a predetermined size based on the detected position of the tumor cell and the detected position of the non-tumor cell is provided.

### Advantageous Effects of Invention

According to an aspect of the present invention, the computational cost for identifying a region having a high tumor cell content rate for the genetic panel test can be reduced.

### BRIEF DESCRIPTION OF DRAWINGS

FIG. 1 is a diagram illustrating an example of transmission and reception of data in a system including a terminal of a pathologist and a server.

FIG. 2 is a block diagram illustrating a configuration of the inspection assistance device according to the first example embodiment.

FIG. 3 is a diagram schematically illustrating an example of image data of a pathology specimen.

FIG. 4 is an example illustrating a screen of a terminal of a pathologist, in which information indicating a tumor cell content rate for each divided region is added on one piece of image data of a pathology specimen.

FIG. 5 is a flowchart illustrating an operation of the inspection assistance device according to the first example embodiment.

FIG. 6 is a block diagram illustrating a configuration of the inspection assistance device according to the second example embodiment.

FIG. 7 is an example illustrating a screen of a terminal of a pathologist, and is a diagram schematically illustrating positions and the number of non-tumor cells and tumor cells in a divided region.

FIG. 8 is a flowchart illustrating an operation of the inspection assistance device according to the second example embodiment.

FIG. 9 is a block diagram illustrating a configuration of the inspection assistance device according to the third example embodiment.

FIG. 10 is an example illustrating a screen of a terminal of a pathologist, and is a diagram illustrating an example of an additionally designated region.

FIG. 11 is a flowchart illustrating an operation of an inspection assistance device according to the third example embodiment.

FIG. 12 is a block diagram illustrating a configuration of an inspection assistance device according to the fourth example embodiment.

FIG. 13 is a diagram illustrating an example of a hardware configuration of the inspection assistance device according to any of the first, second, third, and fourth example embodiments.

### EXAMPLE EMBODIMENT

Example embodiments of the present invention will be described with reference to the drawings.

### (Inspection Assistance System 1)

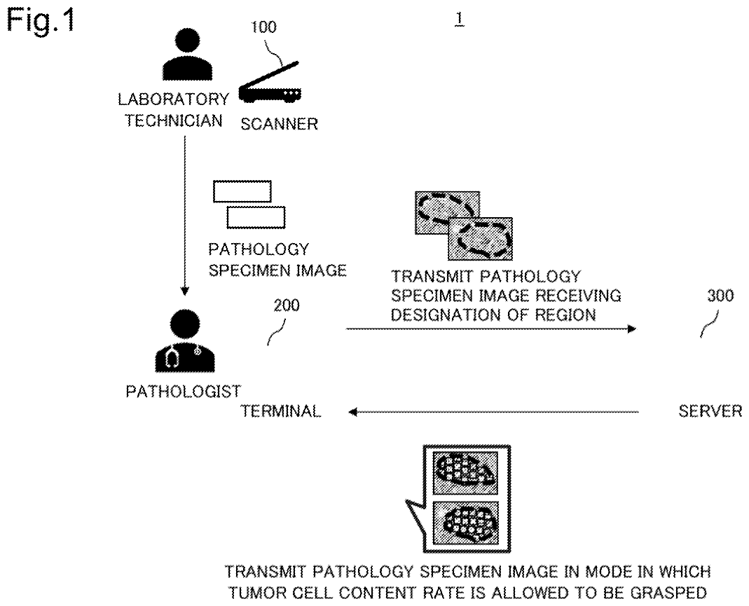

An example of a configuration of an inspection assistance system 1 to which an inspection assistance device 10 according to the first example embodiment to be described later is applied will be described with reference to FIG. 1. FIG. 1 is a diagram schematically illustrating an example of a configuration of a system.

As illustrated in FIG. 1, the inspection assistance system 1 includes a scanner 100 of a laboratory technician, a terminal 200 of a pathologist, and a server 300. The scanner 100 of the laboratory technician and the terminal 200 of the pathologist may be mounted on the same terminal.

The laboratory technician creates a pathology specimen of cellular tissue to be subjected to a genetic test. Specifically, for example, the laboratory technician creates image data (hereinafter, a “pathology specimen image”) obtained by scanning the pathology specimen using the scanner 100. The created pathology specimen image is transmitted to the terminal 200 of the pathologist.

The pathologist designates a region where the calculation processing of the tumor cell content rate is performed on the pathology specimen image transmitted to the terminal 200. The terminal 200 transmits the pathology specimen image and the information indicating the designated region to the server 300. A specific method of designating the region will be described later.

The server 300 calculates the tumor cell content rate for each divided region in the designated region of the pathology specimen image received from the terminal 200. Details of the calculation processing of the tumor cell content rate for each divided region will be described later.

The server 300 transmits the pathology specimen image to the terminal 200 in a mode in which the calculated tumor cell content rate for each divided region is allowed to be grasped. Details of a mode in which the tumor cell content rate is allowed to be grasped will be described later.

In the example embodiment described below, the server 300 will be described as inspection assistance devices 10, 20, and 30.

### First Example Embodiment

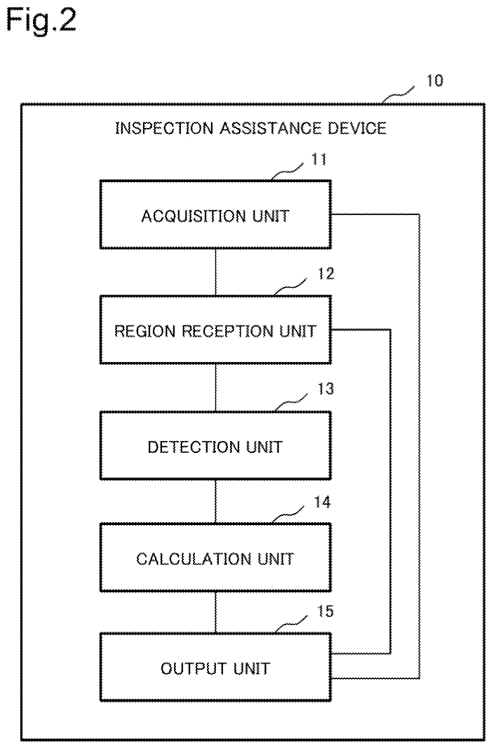

The first example embodiment will be described with reference to FIGS. 2 to 5.

### (Inspection Assistance Device 10)

With reference to FIG. 2, components included in the inspection assistance device 10 according to the first present example embodiment will be described. FIG. 2 is a block diagram illustrating a configuration of the inspection assistance device 10. As illustrated in FIG. 2, the inspection assistance device 10 includes an acquisition unit 11, a region reception unit 12, a detection unit 13, a calculation unit 14, and an output unit 15.

The acquisition unit 11 is an acquisition means configured to acquire an image of the pathology specimen. In an example, the acquisition unit 11 acquires the pathology specimen image transmitted from the terminal 200 (FIG. 1) to the server 300 (FIG. 1). The acquisition unit 11 outputs the acquired pathology specimen image to the region reception unit 12 and the output unit 15.

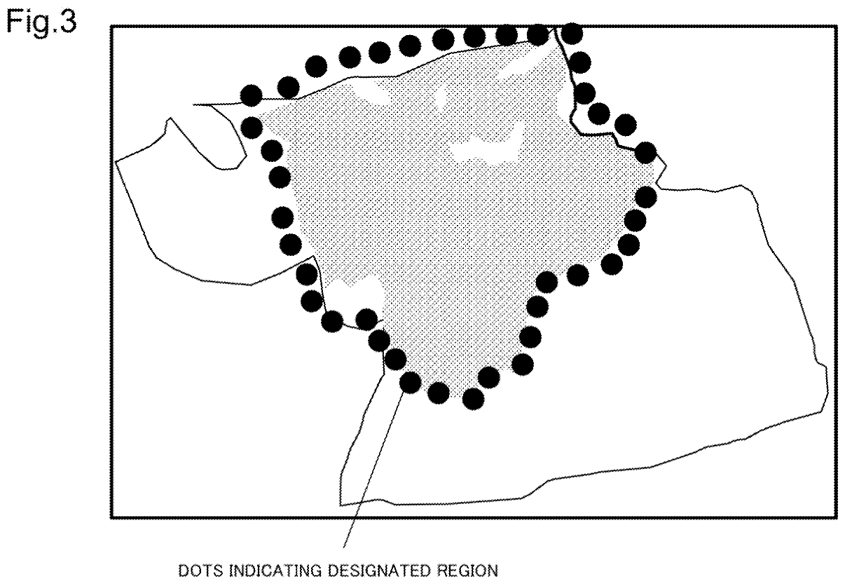

The region reception unit 12 is a region reception means configured to receive designation of a region for the acquired pathology specimen image. The region reception unit 12 receives designation of a region to be processed by the detection unit 13 described later on the pathology specimen image input from the acquisition unit 11. The designation of the region is performed, for example, by a pathologist inputting dots or lines using general image editing software operating on the terminal 200 (FIG. 1). The region inside the input dots or lines is a designated region, that is, a region to be processed by the detection unit 13 described later. FIG. 3 is an example of a pathology specimen image in which a region is designated by dots on the terminal 200.

The designation of the region is not limited to the above, a pathology specimen in which a pathologist draws dots or lines with a magic pen or the like may be read by the scanner 100. In this case, the region reception unit 12 receives dots or lines recognized from the image read by the scanner 100 as designation of the region.

The region reception unit 12 outputs the pathology specimen image input from the acquisition unit 11 and information indicating a region designated for the pathology specimen image to the detection unit 13 and the output unit 15. The information indicating the designated region is, for example, information about coordinates in the pathology specimen image.

The detection unit 13 is a detection means configured to detect the position of the tumor cell and the position of the non-tumor cell in the pathology specimen image in the designated region. The detection unit 13 detects the position of the tumor cell and the position of the non-tumor cell in the pathology specimen image of the designated region based on the pathology specimen image and the information indicating the designated region with respect to the pathology specimen image input from the region reception unit 12. Specifically, the detection unit 13 detects the position of the tumor cell and the position of the non-tumor cell from the pathology specimen image in the designated region using a trained model trained so as to output the position of the tumor cell and the position of the non-tumor cell from the pathology specimen image. For example, the detection unit 13 inputs the pathology specimen image of the designated region to an identifier that has been machine trained on the image characteristics of the tumor cell and the image characteristics of the non-tumor cell, and identifies the tumor cell and the non-tumor cell, thereby detecting the position of the tumor cell and the position of the non-tumor cell in the designated region. The identifier used by the detection unit 13 is not limited to an identifier that has been machine trained, and may be any identifier that can detect tumor cells and non-tumor cells. The detection unit 13 outputs the pathology specimen image of the designated region, the information indicating the position of the tumor cell, and the information indicating the position of the non-tumor cell to the calculation unit 14. The information about the position of the tumor cell and the position of the non-tumor cell is, for example, information about coordinates in the pathology specimen image.

The calculation unit 14 is a calculation means configured to calculate the tumor cell content rate for each divided region obtained by division by a predetermined size based on the detected position of the tumor cell and the detected position of the non-tumor cell. The calculation unit 14 calculates the tumor cell content rate for each divided region obtained by division by a predetermined size based on the pathology specimen image of the designated region, the information indicating the position of the tumor cell, and the information indicating the position of the non-tumor cell input from the detection unit 13. The divided region is, for example, a region obtained by dividing the pathology specimen image of the designated region into rectangles having the same size. The divided region is not limited to a rectangle having the same size, and may be a region obtained by dividing the pathology specimen image of the designated region based on a certain rule. The tumor cell content rate is a ratio of the number of tumor cells to the total number of tumor cells and non-tumor cells obtained from the positions of tumor cells and the positions of non-tumor cells in each divided region. The calculation unit 14 outputs the tumor cell content rate for each divided region to the output unit 15.

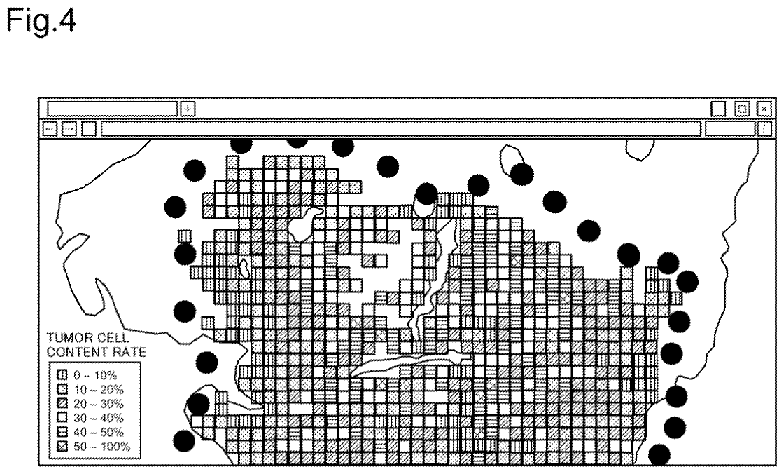

The output unit 15 is an output means configured to output the pathology specimen image in a mode in which the tumor cell content rate for each divided region is allowed to be grasped. The output unit 15 outputs the pathology specimen image to the terminal 200 in a mode in which the region tumor cell content rate for each of the divided regions is allowed to be grasped based on the pathology specimen image input from the acquisition unit 11, the information indicating the region designated for the pathology specimen image input from the region reception unit 12, and the tumor cell content rate for each divided region input from the calculation unit 14. For example, the output unit 15 outputs a pathology specimen image indicating the tumor cell content rate for each divided region by a heat map. FIG. 4 is a diagram illustrating a pathology specimen image indicating the tumor cell content rate for each divided region by a heat map, displayed on the screen of the terminal 200. In FIG. 4, the tumor cell content rate for each divided region is displayed in different patterns classified by 10%. The display of the tumor cell content rate for each divided region is not limited to a different pattern, and it is sufficient that the classification to which the divided region belongs can be visually recognized by a difference in color or the like.

The output unit 15 may be configured in the terminal 200. In this case, the output unit 15 may output the pathology specimen image in a mode in which the tumor cell content rate for each of the divided regions is allowed to be grasped based on the pathology specimen image and the information indicating the designated region stored in the terminal 200, and the tumor cell content rate for each divided region input from the calculation unit 14.

### (Operation of Inspection Assistance Device 10)

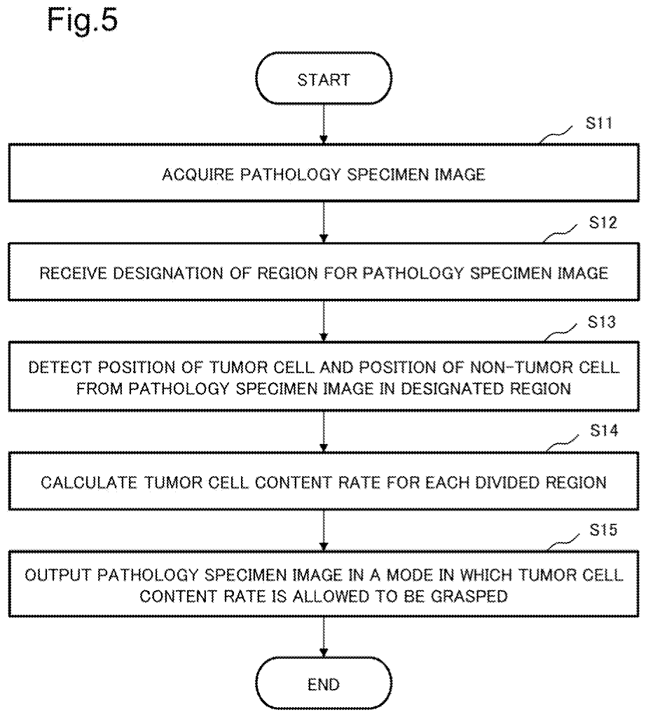

The operation of the inspection assistance device 10 according to the first present example embodiment will be described with reference to FIG. 5. FIG. 5 is a flowchart illustrating a flow of execution number processing by each unit of the inspection assistance device 10.

As illustrated in FIG. 5, the acquisition unit 11 acquires a pathology specimen image (S11). The region reception unit 12 receives designation of a region for the pathology specimen image (S12). The detection unit 13 detects the position of the tumor cell and the position of the non-tumor cell from the pathology specimen image in the designated region (S13). The calculation unit 14 calculates the tumor cell content rate for each divided region based on the positions of the tumor cells and the positions of the non-tumor cells (S14). The output unit 15 outputs the pathology specimen image in a mode in which the tumor cell content rate is allowed to be grasped (S15).

According to the configuration of the present example embodiment, since the tumor cell content rate in the designated region of the pathology specimen image is calculated, the calculation cost for identifying the region having a high tumor cell content rate for the genetic panel test can be reduced as compared with the case of calculating the tumor cell content rates of all the regions of the pathology specimen image.

### Second Example Embodiment

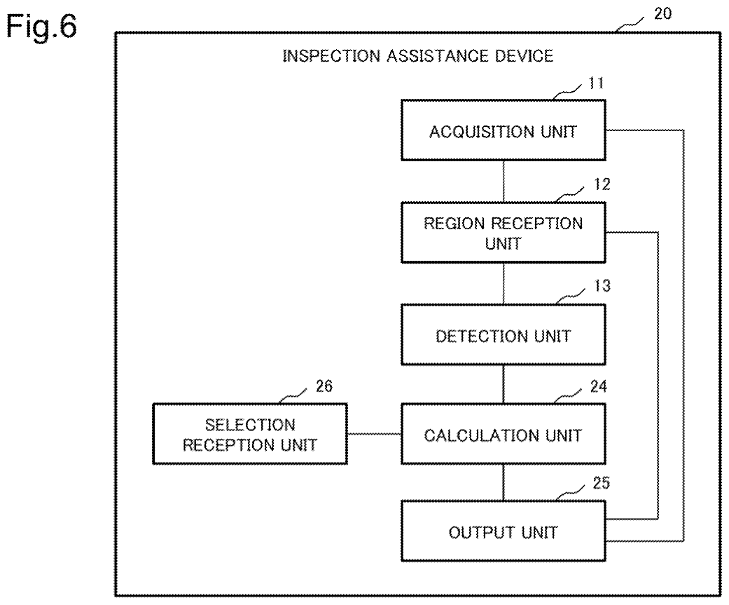

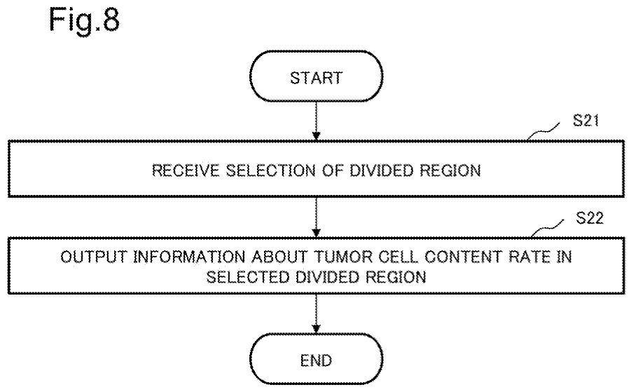

The second example embodiment will be described with reference to FIGS. 6 to 8. In addition to the operation in the first example embodiment, in the second example embodiment, it is possible to display detailed information about the tumor cell content rate of the selected divided region.

### (Inspection Assistance Device 20)

With reference to FIG. 6, components included in the inspection assistance device 20 according to the second present example embodiment will be described. Among the configurations of the inspection assistance device 20, a configuration that performs the same processing operation as the configuration of the inspection assistance device 10 of the first example embodiment is denoted by the same reference numeral as that in FIG. 2, and a detailed description thereof is omitted. FIG. 6 is a block diagram illustrating a configuration of the inspection assistance device 20. As illustrated in FIG. 6, the inspection assistance device 20 includes an acquisition unit 11, a region reception unit 12, a detection unit 13, a calculation unit 24, an output unit 25, and a selection reception unit 26.

The selection reception unit 26 is a selection reception means configured to receive selection of a divided region. For example, the selection reception unit 26 receives selection of the divided region via the screen (FIG. 4) of the terminal 200 that displays the tumor cell content rate for each divided region. The selection reception unit 26 outputs the received selection information about the divided region to the calculation unit 24.

The calculation unit 24 outputs information about the tumor cell content rate in the divided region identified by the selection information input from the selection reception unit 26 to the output unit 25. The information about the tumor cell content rate includes, for example, the position of the tumor cell, the position of the non-tumor cell, the number of tumor cells, the number of non-tumor cells, and the tumor cell content rate. The position of the tumor cell and the position of the non-tumor cell are input from the detection unit 13. The number of tumor cells, the number of non-tumor cells, and the tumor cell content rate are calculated by the same method as in the first example embodiment.

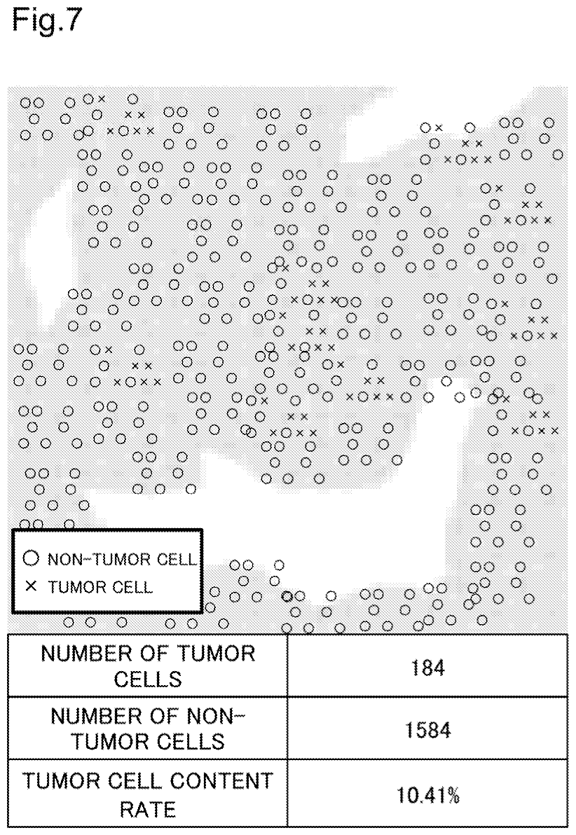

The output unit 25 outputs the information about the tumor cell content rate in the divided region input from the calculation unit 24 to the terminal 200. FIG. 7 is a diagram illustrating an image that can check information about the tumor cell content rate in the selected divided region displayed on the terminal 200. In FIG. 7, the positions of the tumor cells and the positions of the non-tumor cells are schematically superimposed on the pathology specimen image, and the number of tumor cells, the number of non-tumor cells, and the tumor cell content rate are displayed. The output unit 25 does not need to output all of the position of the tumor cell, the position of the non-tumor cell, the number of tumor cells, the number of non-tumor cells, the tumor cell content rate, and the pathology specimen image, and may output them in any combination.

When the divided region is pointed on the screen of the terminal 200 illustrated in FIG. 4, the output unit 25 may output at least one of the number of tumor cells, the number of non-tumor cells, and the tumor cell content rate in the pointed divided region in such a way that it is superimposed and displayed on the screen of the terminal 200 illustrated in FIG. 4. The output unit 25 may be configured in the terminal 200.

### (Operation of Inspection Assistance Device 20)

The selection reception unit 26 receives selection of a divided region (S16). The output unit 25 outputs information about the tumor cell content rate in the selected divided region (S17).

According to the configuration of the present example embodiment, a pathologist or a laboratory technician can check detailed information such as the position of the tumor cell and the number of tumor cells in the divided region as necessary.

### Third Example Embodiment

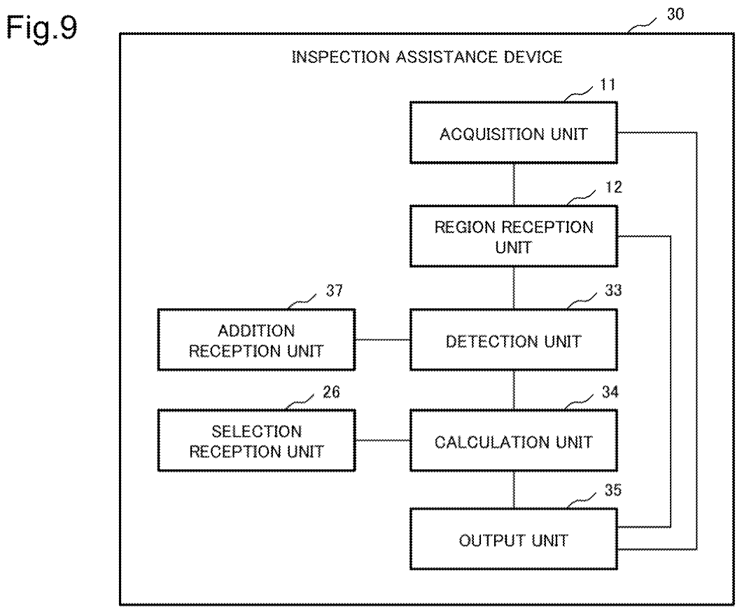

The third example embodiment will be described with reference to FIGS. 9 to 11. In addition to the operation in the first example embodiment, in the third example embodiment, it is possible to add a region for calculating the tumor cell content rate other than the region where the tumor cell content rate was calculated.

### (Inspection Assistance Device 30)

With reference to FIG. 9, components included in the inspection assistance device 30 according to the third present example embodiment will be described. Among the configurations of the inspection assistance device 30, a configuration that performs the same processing operation as the configuration of the inspection assistance device 20 of the second example embodiment is denoted by the same reference numeral as that in FIG. 6, and a detailed description thereof is omitted. FIG. 9 is a block diagram illustrating a configuration of the inspection assistance device 30. As illustrated in FIG. 9, the inspection assistance device 30 includes an acquisition unit 11, a region reception unit 12, a detection unit 33, a calculation unit 34, an output unit 35, a selection reception unit 26, and an addition reception unit 37.

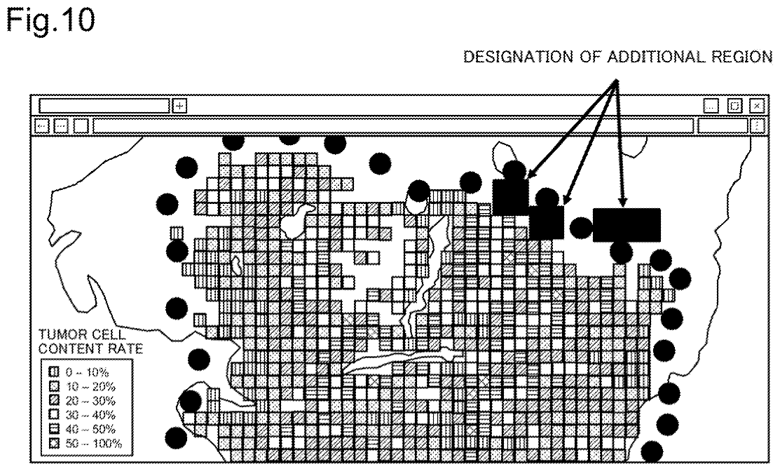

The addition reception unit 37 is an addition reception means configured to receive designation of an additional region (hereinafter, referred to as an “additional region”) other than the divided region for which the tumor cell content rate has been calculated. For example, the addition reception unit 37 receives designation of the additional region via the screen of the terminal 200 on which the pathology specimen image is displayed. FIG. 10 illustrates an example of a screen displayed by the terminal 200 that receives designation of the additional region. The designation of the additional region is only required to be able to identify the region, and for example, a rectangular region is selected as illustrated in FIG. 10. The addition reception unit 37 outputs information about the designated additional region to the detection unit 33.

The detection unit 33 detects the position of the tumor cell and the position of the non-tumor cell from the pathology specimen image in the additional region based on the information about the additional region received by the addition reception unit 37. The position of the tumor cell and the position of the non-tumor cell are detected by the method same as that of the detection unit 13 of the first example embodiment. The detection unit 33 outputs the pathology specimen image of the additional region, the information about the position of the tumor cell in the additional region, and the information about the position of the non-tumor cell to the calculation unit 34.

The calculation unit 34 calculates the tumor cell content rate for each divided region obtained by dividing the additional region by a predetermined size based on the pathology specimen image of the additional region, the information about the position of the tumor cell in the additional region, and the information about the position of the non-tumor cell input from the detection unit 33. The tumor cell content rate is calculated by the method same as that of the calculation unit 14 of the first example embodiment. In a case where the size of the additional region is substantially the same as the size of the divided region, the calculation unit 34 may calculate the tumor cell content rate as the divided region without dividing the added region. The calculation unit 34 outputs the tumor cell content rate for each divided region in the additional region to the output unit 35.

The output unit 35 outputs the pathology specimen image to the terminal 200 in a mode in which the tumor cell content rate in the additional region is allowed to be grasped based on the pathology specimen image input from the acquisition unit 11 and the tumor cell content rate for each divided region in the additional region input from the calculation unit 34. A mode in which the tumor cell content rate in the additional region is allowed to be grasped is the same as the mode described in the first example embodiment.

The output unit 35 may output a region expected to have a high tumor cell content rate other than the divided region on the screen of the terminal 200 as a recommended additional region based on the calculation result of the tumor cell content rate by the calculation unit 34. As a result, the pathologist or the laboratory technician can designate the additional region with reference to the screen of the terminal 200 on which the recommended additional region is displayed.

### (Operation of Inspection Assistance Device 30)

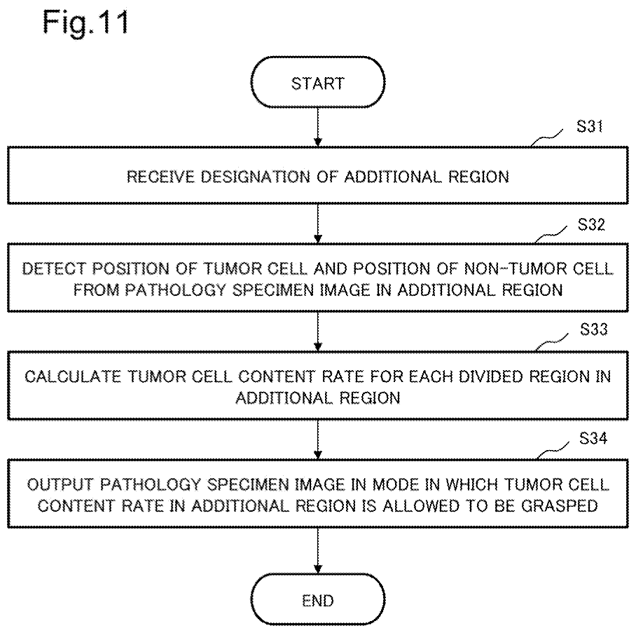

The operation of the inspection assistance device 30 according to the third present example embodiment will be described with reference to FIG. 11. FIG. 11 is a flowchart illustrating a flow of execution number processing by each unit of the inspection assistance device 30.

As illustrated in FIG. 11, the addition reception unit 37 receives designation of an additional region (S31). The detection unit 33 detects the position of the tumor cell and the position of the non-tumor cell from the pathology specimen image in the additional region (S32). The calculation unit 34 calculates the tumor cell content rate for each divided region in the additional region (S33). The output unit 35 outputs the pathology specimen image in a mode in which the tumor cell content rate in the additional region is allowed to be grasped (S34).

According to the configuration of the present example embodiment, the pathologist or the laboratory technician can designate a region where the tumor cell content rate is desired to be additionally checked. For example, when a divided region with a high tumor cell content rate is close to the edge of the initially designated region, it may be desired to additionally check the tumor cell content rate of the outer region close to the edge of the region. For example, even in the initially designated region, the size is less than the size of the divided region, and the tumor cell content rate of the region to be checked may not be calculated. In such a case, according to the configuration of the present example embodiment, it is possible to additionally calculate the tumor cell content rate of the region to be checked.

### Fourth Example Embodiment

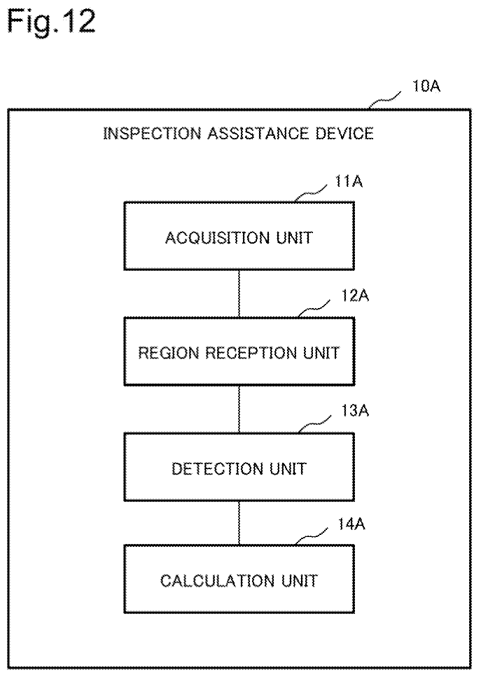

The fourth example embodiment will be described with reference to FIG. 12.

### (Inspection Assistance Device 10A)

With reference to FIG. 12, components included in an inspection assistance device 10A according to the fourth present example embodiment will be described. For example, an acquisition unit 11A, a region reception unit 12A, a detection unit 13A, and a calculation unit 14A can be configured as in the acquisition unit 11, the region reception unit 12, the detection unit 13, and the calculation unit 14, respectively, but the present invention is not limited thereto.

The acquisition unit 11A acquires a pathology specimen image to output the pathology specimen image to the region reception unit 12A. The region reception unit 12A receives designation of a region with respect to the acquired pathology specimen image, to output the region to a detection unit 13A. The detection unit 13A detects the position of the tumor cell and the position of the non-tumor cell from the pathology specimen image in the designated region to output the positions to the calculation unit 14. The calculation unit 14 calculates the tumor cell content rate for each divided region obtained by division by a predetermined size based on the detected position of the tumor cell and the detected position of the non-tumor cell.

Also according to the fourth example embodiment, the inspection assistance device 10A can reduce the calculation cost for identifying a region having a high tumor cell content rate for the genetic panel test.

### (Hardware Configuration)

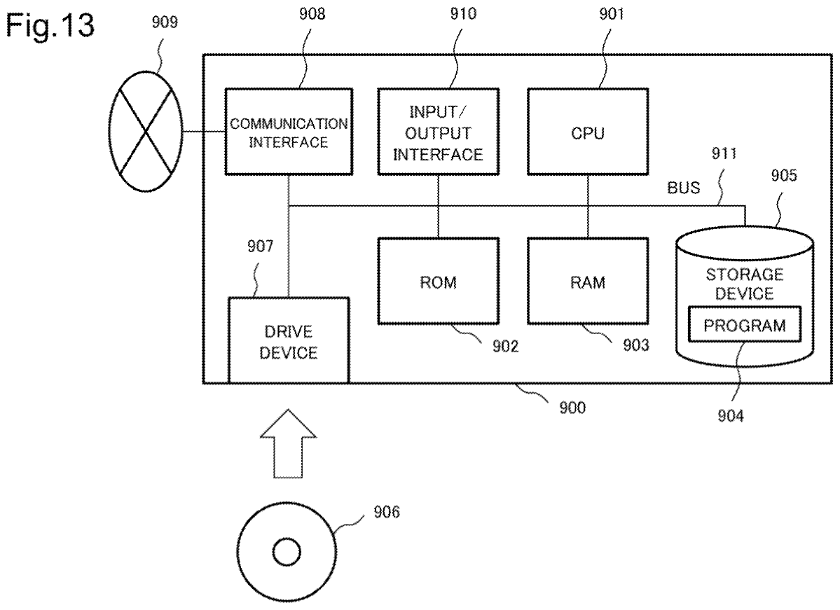

Each component of the inspection assistance device 10, the inspection assistance device 20, the inspection assistance device 30, and the inspection assistance device 10A described in the first, second, third, and fourth example embodiments, respectively, represents a block of a functional unit. Some or all of these components are implemented by an information processing device 900 as illustrated in FIG. 13, for example. FIG. 13 is a block diagram illustrating an example of a hardware configuration of the information processing device 900.

As illustrated in FIG. 13 the information processing device 900 includes the following configuration as an example.
central processing unit (CPU) 901
read only memory (ROM) 902
random access memory (RAM) 903
program 904 loaded into RAM 903
storage device 905 storing program 904
drive device 907 that reads and writes recording medium 906
communication interface 908 connected to a communication network 909
input/output interface 910 for inputting/outputting data
bus 911 connecting respective components

The components of the inspection assistance device 10, the inspection assistance device 20, the inspection assistance device 30, and the inspection assistance device 10A described in the first, second, third, and fourth example embodiments are implemented by the CPU 901 reading and executing the program 904 that implements these functions. The program 904 for achieving the function of each component is stored in the storage device 905 or the ROM 902 in advance, for example, and the CPU 901 loads the program into the RAM 903 and executes the program as necessary. The program 904 may be supplied to the CPU 901 via the communication network 909, or may be stored in advance in the recording medium 906, and the drive device 907 may read the program and supply the program to the CPU 901.

According to the above configuration, the inspection assistance device 10, the inspection assistance device 20, the inspection assistance device 30, and the inspection assistance device 10A described in the first, second, third, and fourth example embodiments are achieved as hardware. Therefore, effects similar to the effects described in the above example embodiment can be obtained.

Although the present invention is described with reference to the respective example embodiments (and examples), the present invention is not limited to the above example embodiments (and examples). Various modifications that can be understood by those skilled in the art can be made to the configurations and details of the above example embodiments (and examples) within the scope of the present invention. For example, the configurations of the second example embodiment and the third example embodiment can be combined.

Some or all of the above example embodiments may be described as the following Supplementary Notes, but are not limited to the following.

### (Supplementary Note 1)

An inspection assistance device including
an acquisition means configured to acquire a pathology specimen image,
a region reception means configured to receive designation of a region for the acquired pathology specimen image,
a detection means configured to detect a position of a tumor cell and a position of a non-tumor cell in the pathology specimen image in the designated region, and
a calculation means configured to calculate a tumor cell content rate for each divided region obtained by division by a predetermined size based on the detected position of the tumor cell and the detected position of the non-tumor cell.

### (Supplementary Note 2)

The inspection assistance device according to Supplementary Note 1, further including
an output means configured to output the pathology specimen image in a mode in which the tumor cell content rate for the each divided region is allowed to be grasped.

### (Supplementary Note 3)

The inspection assistance device according to Supplementary Note 2, wherein
the output means outputs the pathology specimen image indicating the tumor cell content rate for the each divided regions by a heat map.

### (Supplementary Note 4)

The inspection assistance device according to Supplementary Note 2 or 3, further including
a selection reception means configured to receive selection of the divided region, wherein
the output means outputs information about the tumor cell content rate in the divided region whose selection was received.

### (Supplementary Note 5)

The inspection assistance device according to Supplementary Note 4, wherein
the output means outputs, as information about the tumor cell content rate, information indicating at least one of the position of the tumor cell and the position of the non-tumor cell in the divided region whose selection was received.

### (Supplementary Note 6)

The inspection assistance device according to Supplementary Note 4 or 5, wherein
the output means outputs, as information about the tumor cell content rate, information indicating at least one of the number of tumor cells and the number of non-tumor cells in the divided region whose selection was received.

### (Supplementary Note 7)

The inspection assistance device according to any one of Supplementary Notes 4 to 6, further including
an addition reception means configured to receive designation of an additional region other than the divided region, wherein
the detection means detects a position of a tumor cell and a position of a non-tumor cell from the pathology specimen image in the designated additional region, and wherein
the calculation means calculates a tumor cell content rate for each divided region obtained by division by a predetermined size based on the detected position of the tumor cell and the detected position of the non-tumor cell in the additional region.

### (Supplementary Note 8)

The inspection assistance device according to Supplementary Note 7, wherein
the output means outputs the pathology specimen image in a mode in which the tumor cell content rate for the each divided region in the additional region is allowed to be grasped.

### (Supplementary Note 9)

The inspection assistance device according to any one of Supplementary Notes 1 to 8, wherein
the detection means detects, using a trained model trained so as to output a position of a tumor cell and a position of a non-tumor cell from a pathology specimen image, a position of a tumor cell and a position of a non-tumor cell from the pathology specimen image in the designated region.

### (Supplementary Note 10)

An inspection assistance system including
an acquisition means configured to acquire a pathology specimen image,
a region reception means configured to receive designation of a region for the acquired pathology specimen image,
a detection means configured to detect a position of a tumor cell and a position of a non-tumor cell in the pathology specimen image in the designated region, and
a calculation means configured to calculate a tumor cell content rate for each divided region obtained by division by a predetermined size based on the detected position of the tumor cell and the detected position of the non-tumor cell.

### (Supplementary Note 11)

An inspection assistance method including
acquiring a pathology specimen image,
receiving designation of a region for the acquired pathology specimen image,
detecting a position of a tumor cell and a position of a non-tumor cell in the pathology specimen image in the designated region, and
calculating a tumor cell content rate for each divided region obtained by division by a predetermined size based on the detected position of the tumor cell and the detected position of the non-tumor cell.

### (Supplementary Note 12)

The inspection assistance method according to Supplementary Note 11, further including
outputting the pathology specimen image in a mode in which the tumor cell content rate for the each divided region is allowed to be grasped.

### (Supplementary Note 13)

The inspection assistance method according to Supplementary Note 12, wherein
the outputting includes outputting the pathology specimen image indicating the tumor cell content rate for the each divided region by a heat map.

### (Supplementary Note 14)

The inspection assistance method according to Supplementary Note 12 or 13, further including
receiving selection of the divided region, and
outputting information about the tumor cell content rate in the divided region whose selection was received.

### (Supplementary Note 15)

The inspection assistance method according to Supplementary Note 14, wherein
the outputting includes outputting, as information about the tumor cell content rate, information indicating at least one of the position of the tumor cell and the position of the non-tumor cell in the divided region whose selection was received.

### (Supplementary Note 16)

The inspection assistance method according to Supplementary Note 14 or 15, wherein
the outputting includes outputting, as information about the tumor cell content rate, information indicating at least one of the number of tumor cells and the number of non-tumor cells in the divided region whose selection was received.

### (Supplementary Note 17)

The inspection assistance method according to any one of Supplementary Notes 14 to 16, further including
receiving designation of an additional region other than the divided region;
detecting a position of a tumor cell and a position of a non-tumor cell from the pathology specimen image in the designated additional region; and
calculating a tumor cell content rate for each divided region obtained by division by a predetermined size based on the detected position of the tumor cell and the detected position of the non-tumor cell in the additional region.

### (Supplementary Note 18)

The inspection assistance method according to Supplementary Note 17, wherein
the outputting includes outputting the pathology specimen image in a mode in which the tumor cell content rate for the each divided region in the additional region is allowed to be grasped.

### (Supplementary Note 19)

The inspection assistance method according to any one of Supplementary Notes 11 to 18, wherein
the detecting includes detecting, using a trained model trained so as to output a position of a tumor cell and a position of a non-tumor cell from a pathology specimen image, a position of a tumor cell and a position of a non-tumor cell from the pathology specimen image in the designated region.

### (Supplementary Note 20)

A recording medium storing a program for causing a computer to function as
an acquisition means configured to acquire a pathology specimen image,
a region reception means configured to receive designation of a region for the acquired pathology specimen image,
a detection means configured to detect a position of a tumor cell and a position of a non-tumor cell in the pathology specimen image in the designated region, and
a calculation means configured to calculate a tumor cell content rate for each divided region obtained by division by a predetermined size based on the detected position of the tumor cell and the detected position of the non-tumor cell.

### REFERENCE SIGNS LIST

10, 20, 30, 10A inspection assistance device
11, 11A acquisition unit
12, 12A region reception unit
13, 33, 13A detection unit
14, 24, 34, 14A calculation unit
15, 25, 35 output unit
26 selection reception unit
37 addition reception unit
100 scanner
200 terminal
300 server
901 central processing unit (CPU)
902 read only memory (ROM)
903 random access memory (RAM) 903
904 program 904
905 storage device 905
906 recording medium
907 drive device
908 communication interface
909 communication network
910 input/output interface for input/outputting data

What is claimed is:

1. An inspection assistance device comprising:
a memory configured to store instructions; and
at least one processor configured to execute the instructions to perform:
acquiring a pathology specimen image;
receiving designation of a region for the acquired pathology specimen image;
detecting a position of a tumor cell and a position of a non-tumor cell in the pathology specimen image in the designated region; and
calculating a tumor cell content rate for each divided region obtained by division by a predetermined size based on the detected position of the tumor cell and the detected position of the non-tumor cell.

2. The inspection assistance device according to claim 1, wherein
the at least one processor is configured to execute the instructions to perform:
outputting the pathology specimen image in a mode in which the tumor cell content rate for the each divided region is allowed to be grasped.

3. The inspection assistance device according to claim 2, wherein
the at least one processor is configured to execute the instructions to perform:
outputting the pathology specimen image indicating the tumor cell content rate for the each divided regions by a heat map.

4. The inspection assistance device according to claim 2, wherein
the at least one processor is configured to execute the instructions to perform:
receiving selection of the divided region, and
outputting information about the tumor cell content rate in the divided region whose selection was received.

5. The inspection assistance device according to claim 4, wherein
the at least one processor is configured to execute the instructions to perform:
outputting, as information about the tumor cell content rate, information indicating at least one of the position of the tumor cell and the position of the non-tumor cell in the divided region whose selection was received.

6. The inspection assistance device according to claim 4, wherein
the at least one processor is configured to execute the instructions to perform:
outputting, as information about the tumor cell content rate, information indicating at least one of the number of tumor cells and the number of non-tumor cells in the divided region whose selection was received.

7. The inspection assistance device according to claim 4, further comprising:
the at least one processor is configured to execute the instructions to perform:
receiving designation of an additional region other than the divided region,
detecting a position of a tumor cell and a position of a non-tumor cell from the pathology specimen image in the designated additional region, and
calculating a tumor cell content rate for each divided region obtained by division by a predetermined size based on the detected position of the tumor cell and the detected position of the non-tumor cell in the additional region.

8. The inspection assistance device according to claim 7, wherein
the at least one processor is configured to execute the instructions to perform:
outputting the pathology specimen image in a mode in which the tumor cell content rate for the each divided region in the additional region is allowed to be grasped.

9. The inspection assistance device according to claim 1, wherein
the at least one processor is configured to execute the instructions to perform:
detecting, using a trained model trained so as to output a position of a tumor cell and a position of a non-tumor cell from a pathology specimen image, a position of a tumor cell and a position of a non-tumor cell from the pathology specimen image in the designated region.

10. (canceled)

11. An inspection assistance method comprising:
acquiring a pathology specimen image;
receiving designation of a region for the acquired pathology specimen image;
detecting a position of a tumor cell and a position of a non-tumor cell in the pathology specimen image in the designated region; and
calculating a tumor cell content rate for each divided region obtained by division by a predetermined size based on the detected position of the tumor cell and the detected position of the non-tumor cell.

12. The inspection assistance method according to claim 11, further comprising:
outputting the pathology specimen image in a mode in which the tumor cell content rate for the each divided region is allowed to be grasped.

13. The inspection assistance method according to claim 12, wherein
the outputting includes outputting the pathology specimen image indicating the tumor cell content rate for the each divided region by a heat map.

14. The inspection assistance method according to claim 12, further comprising:
receiving selection of the divided region; and
outputting information about the tumor cell content rate in the divided region whose selection was received.

15. The inspection assistance method according to claim 14, wherein
the outputting includes outputting, as information about the tumor cell content rate, information indicating at least one of the position of the tumor cell and the position of the non-tumor cell in the divided region whose selection was received.

16. The inspection assistance method according to claim 14, wherein
the outputting includes outputting, as information about the tumor cell content rate, information indicating at least one of the number of tumor cells and the number of non-tumor cells in the divided region whose selection was received.

17. The inspection assistance method according to claim 14, further comprising:
receiving designation of an additional region other than the divided region;
detecting a position of a tumor cell and a position of a non-tumor cell from the pathology specimen image in the designated additional region; and
calculating a tumor cell content rate for each divided region obtained by division by a predetermined size based on the detected position of the tumor cell and the detected position of the non-tumor cell in the additional region.

18. The inspection assistance method according to claim 17, wherein
the outputting includes outputting the pathology specimen image in a mode in which the tumor cell content rate for the each divided region in the additional region is allowed to be grasped.

19. The inspection assistance method according to claim 11, wherein
the detecting includes detecting, using a trained model trained so as to output a position of a tumor cell and a position of a non-tumor cell from a pathology specimen image, the position of the tumor cell and the position of the non-tumor cell from the pathology specimen image in the designated region.

20. A non-transitory recording medium storing a program for causing a computer to perform:
acquiring a pathology specimen image;
receiving designation of a region for the acquired pathology specimen image;
detecting a position of a tumor cell and a position of a non-tumor cell in the pathology specimen image in the designated region; and
calculating a tumor cell content rate for each divided region obtained by division by a predetermined size based on the detected position of the tumor cell and the detected position of the non-tumor cell.
